(19) United States
(12) Patent Application Publication
(10) Pub. No.: US 2023/0276842 A1
(43) Pub. Date: Sep. 7, 2023

(54) HYGIENE MOUTHPIECE ELEMENT, HANDLE MOUTHPIECE ELEMENT, TUBE DEVICE FOR WATER PIPES AND WATER PIPE

(71) Applicant: Toufic Hafez El Riz, Lüneburg (DE)

(72) Inventor: Toufic Hafez El Riz, Lüneburg (DE)

(73) Assignee: Toufic Hafez El Riz, Lüneburg (DE)

(21) Appl. No.: 18/024,167

(22) PCT Filed: Sep. 1, 2021
(86) PCT No.: PCT/EP2021/074132

§ 371 Date: Mar. 1, 2023

(30) Foreign Application Priority Data
Sep. 1, 2020 (DE) 20 2020 105 028.1

(51) Int. Cl. A24F 7/04; A24F 1/30; A24F 7/02; A24D 3/16; A24D 3/06; A24D 3/04; A61L 2/232; A61L 2/238

(52) U.S. Cl. CPC A24F 7/04; A24F 1/30; A24F 7/02; A24D 3/163; A24D 3/16; A24D 3/063; A24D 3/04; A61L 2/232; A61L 2/238; A61L 2202/24

(57) ABSTRACT

In order to provide a hygiene mouthpiece element which makes it possible to reliably prevent the transmission of micro-organisms, in particular fungi and bacteria, and viruses when using a water pipe, a hygiene mouthpiece element is proposed, comprising a first end section which is configured for connection to a handle mouthpiece element of a water pipe and comprising a second end section which is configured as a mouth-side free end, wherein the hygiene mouthpiece element comprises a first filter element, wherein the first filter element comprises at least one adsorbing material and/or wherein the hygiene mouthpiece element comprises a second filter element, wherein the second filter element comprises metal oxide and/or electrolytically charged fibres.

### CROSS REFERENCE TO RELATED APPLICATIONS

This application is a national stage of International Application No. PCT/EP2021/074132 filed on Sep. 1, 2021, which claims priority to DE Patent Application No. 202020105028.1 filed on Sep. 1, 2020, all of which are hereby incorporated by reference in their entireties.

The invention relates to a hygiene mouthpiece element, a handle mouthpiece element, a mouthpiece device comprising such a hygiene mouthpiece element and/or such a handle mouthpiece element, a tube device as well as water pipes, inter alia comprising such a mouthpiece device or such a tube device.

Water pipes, also called shishas, are frequently used jointly by a group of people sitting together, where one water pipe is used successively by several people. As a result of the joint smoking of one water pipe, this is a source of infection for numerous diseases, which are transmitted by micro-organisms, in particular fungi and bacteria, and viruses. These diseases include, for example, tuberculosis, hepatitis, pneumonia and influenza.

As a result of the spread of the new type of corona virus SARS-CoV-2 and the disease COVID-19 caused by this virus, in particular operators of shisha cafes and bars are faced with the problem that the hygiene required to curb the corona virus SARS-CoV-2 cannot be adhered to by offering water pipes for joint smoking.

In order to increase the hygiene when a water pipe is smoked jointly by several people, so-called hygiene mouthpieces are known from the prior art, which are frequently configured as disposable articles and can be changed when the water pipe is used by another person. Although such hygiene mouthpieces offer a certain protection, such hygiene mouthpieces cannot reliably prevent micro-organisms, in particular fungi and bacteria, and viruses from penetrating into the water pipe.

It is further known to arrange filters inside the water pipe which filter the smoke inside the water pipe. However, such filters usually remain in the water pipe whilst this is used by several people. Thus, even with this solution, it cannot be eliminated that micro-organisms, in particular fungi and bacteria, and viruses are transmitted from one person to another person via the water pipe if the filter is not changed when the water pipe is passed on.

Another problem is that micro-organisms, in particular fungi and bacteria, and viruses can not only be transmitted via respiratory air but that micro-organisms and viruses can also be located on the hands of the persons so that micro-organisms and viruses can also be transmitted from one person to another via the surface of the water pipe.

It is therefore the object of the present invention to provide a water pipe as well as a mouthpiece device for a water pipe which makes it possible to reliably prevent the transmission of micro-organisms, in particular fungi and bacteria, and viruses when using a water pipe. Furthermore, it is the object of the invention to provide a hygiene mouthpiece element and a handle mouthpiece element for such a mouthpiece device or water pipe.

The object according to the invention is achieved by a hygiene mouthpiece element comprising a first end section which is configured for connection to a handle mouthpiece element of a water pipe and comprising a second end section which is configured as a mouth-side free end. The hygiene mouthpiece element comprises a first filter element, wherein the first filter element comprises at least one adsorbing material and/or wherein the hygiene mouthpiece element comprises a second filter element, wherein the second filter element comprises metal oxide and/or electrolytically charged fibres.

Preferably the hygiene mouthpiece element comprises an attachment for mouthpiece devices for water pipes, which can be optionally fastened to a handle mouthpiece element of a water pipe. In particular, the hygiene mouthpiece element preferably comprises a disposable article which is removed by a person after use and is optionally replaced by a new hygiene mouthpiece element before further use by another person. The hygiene mouthpiece element can preferably be used by one person several times.

The hygiene mouthpiece element therefore preferably comprises plastic or is made of plastic. The hygiene mouthpiece element can therefore particularly preferably be manufactured easily and cheaply. If the hygiene mouthpiece element is assigned to one person and used many times by this person, the mouthpiece element can preferably be made of other materials, for example, glass, so that this is designed for longer use.

The first filter element and/or the second filter element are preferably arranged in the hygiene mouthpiece element in such a manner that this or these cover the entire cross-section of the passage through which the smoke drawn in by the water pipe passes. On the one hand, contaminants are thereby advantageously filtered from the smoke so that the smoking person does not breath this in and on the other hand, it is further advantageously prevented that micro-organisms, in particular fungi and bacteria, and viruses can penetrate from the respiratory air of the persons further into the interior of the water pipe since the micro-organisms, in particular fungi and bacteria, and viruses are already held in the hygiene mouthpiece element by means of the first filter element or the second filter element.

Preferably in particular the combination of the first filter element and the second filter element in the hygiene mouthpiece element enable a particularly effective filtering of micro-organisms, in particular fungi and bacteria, and viruses.

Preferably the first filter element and/or the second filter element are configured as fibre layers. As a result of the in particular flat configuration of the two filter elements, the smoke can easily penetrate the first filter element and/or the second filter element in particular transversely to the surface of the fibre layer whilst at the same time an effective filtering of the drawn-in smoke or the respiratory air can be accomplished.

Preferably the electrostatically charged fibres have a fibre diameter of 1 μm to 4 μm, particularly preferably 1.5 μm to 3 μm, quite particularly preferably 2 μm. With the aid of such fibres, the filtering of micro-organisms, in particular fungi and bacteria, and viruses is advantageously possible with an efficiency of preferably at least 99%, particularly preferably at least 99.925%, further particularly preferably 99.99%, quite particularly preferably 99.999999%.

Preferably the first filter element and/or the second filter element comprises a fibre product produced in the meltblown method. Such a fibre product is particularly preferably configured to be particularly thin with very high filter efficiency at the same time.

Preferably the adsorbing material of the first filter element is activated charcoal, in particular activated charcoal fibres. Preferably the activated charcoal is arranged in granular form in the first filter element. Furthermore, it can preferably be provided that the activated charcoal is present in powder form.

Alternatively, the adsorbing material of the first filter element can particularly preferably comprise any material that is capable of adsorbing micro-organisms, in particular fungi and bacteria, and viruses. The adsorbing material can, for example, comprise inter alia zeolite or further known adsorbents. In particular, the adsorbing material is capable of adsorbing corona viruses such as SARS-CoV-2.

Preferably the adsorbing material of the first filter element comprises metal oxide. The metal oxide advantageously increases the adsorption efficiency of the first filter element. Preferably the metal oxide is arranged in granular form in the first filter element. Further preferably, an at least partial coating of the activated charcoal with metal oxide can be provided.

In particular, preferably a particularly effective adsorption or filtering of micro-organisms, in particular fungi and bacteria, and viruses in the first filter element is achieved by the combination of activated charcoal and metal oxide in the first filter element. Particularly preferably both the activated charcoal and also the metal oxide can be provided in granular form in the first filter element.

Preferably the first filter element and/or the second filter element comprises copper oxide. Copper oxide enables micro-organisms, in particular fungi and bacteria, and viruses to be killed or deactivated particularly effectively.

Preferably the first filter element and/or the second filter element comprises a fibre product, in particular a nanofibre product.

Particularly preferably the second filter element comprises nanofibres charged with metal oxide, in particular copper oxide. This enables micro-organisms, in particular fungi and bacteria, and viruses to be killed or deactivated particularly effectively.

Fundamentally the first and/or the second filter element can preferably comprise all materials which can be charged with metal oxide, in particular copper oxide.

Preferably, in addition to the first filter element and the second filter element, the hygiene mouthpiece element comprises further filter elements, wherein the filter elements are preferably arranged one after the other viewed in the flow direction of the drawn-in smoke. Thus, the hygiene mouthpiece element can preferably comprise three, four or even more filter elements. By providing further filter elements, the filter efficiency can advantageously be increased and arbitrarily adapted. Preferably the further filter elements are configured identically to the first filter element and/or the second filter element. Alternatively the further filter elements can be configured differently to the first and/or the second filter element.

For example, the hygiene mouthpiece element can have the first filter element comprising activated charcoal, the second filter element comprising electrostatically charged fibres and a further filter element comprising metal oxide, in particular copper oxide. Further, preferably any other combinations of filter elements and filter technologies are feasible.

Preferably the surface of the hygiene mouthpiece element, in particular a housing of the hygiene mouthpiece element, has a coating in some areas or over the entire area. The coating is configured to kill or deactivate micro-organisms, in particular fungi and bacteria, and viruses, on the surface. Preferably the coating is configured to kill or deactivate at least 50%, particularly preferably at least 75%, quite particularly preferably at least 99.99%, of the micro-organisms, in particular fungi and bacteria, and viruses located on the surface.

In the sense of the present invention, the terms “to kill or deactivate micro-organisms, in particular fungi and bacteria, and viruses” should be understood in such a manner that the micro-organisms, in particular fungi and bacteria, and viruses are impaired in their function to such an extent that diseases can no longer be triggered by them.

The surface of the hygiene mouthpiece element preferably comprises all the areas of the hygiene mouthpiece element facing the surroundings. Particularly preferably the coating is therefore provided on a housing of the hygiene mouthpiece element in which the first filter element and/or the second filter element are arranged. Preferably the surface of the hygiene mouthpiece element also comprises the surfaces of the interior of the hygiene mouthpiece element, in particular the interior of the housing of the hygiene mouthpiece element.

The coating of the surface of the hygiene mouthpiece element advantageously makes it possible to prevent any transmission of micro-organisms, in particular fungi and bacteria, and viruses which are present on the hands of a person and which are therefore transmitted onto the surface of the hygiene mouthpiece element when touching the hygiene mouthpiece element.

The coating can preferably be applied to the surface of the hygiene mouthpiece element during manufacture of the hygiene mouthpiece element. Alternatively the coating of the surface of the hygiene mouthpiece element can also be accomplished subsequently.

Preferably the entire surface of the hygiene mouthpiece element is provided with a coating. Alternatively only one area or several areas of the hygiene mouthpiece element can have the coating. In particular, it is feasible that in particular, a handle region of the hygiene mouthpiece element exclusively has the coating.

Preferably the coating comprises metal oxide ions. Metal oxide ions are able to very effectively kill or deactivate micro-organisms, in particular fungi and bacteria, and viruses.

Preferably the first filter element and the second filter element are arranged one behind the other in the flow direction of smoke sucked through the water pipe. As a result, preferably a first filtering or adsorption is achieved by means of the first or second filter element and subsequently micro-organisms, in particular fungi and bacteria, and viruses which have not been killed or deactivated by means of the first or second filter element, are killed or deactivated by means of the respectively other filter element in a second filtering or adsorption, with the result that the efficiency of the filtering in the water pipe is advantageously increased.

Preferably the first filter element is arranged upstream of the second filter element viewed in the flow direction of smoke sucked through the water pipe. Alternatively the second filter element can preferably be arranged upstream of the first filter element viewed in the flow direction of smoke sucked through the water pipe.

Preferably the first filter element and/or the second filter element are or is arranged non-detachably in the hygiene mouthpiece element. Preferably the first filter element and/or the second filter element are arranged or fastened firmly in the hygiene mouthpiece element. Preferably the first filter element and/or the second filter element are removed together with the hygiene mouthpiece element when this is changed. This is in particular provided in the embodiment of the hygiene mouthpiece element as a disposable article. Since the first filter element and/or the second filter element are arranged non-exchangeably in the hygiene mouthpiece element, the hygiene mouthpiece element can be manufactured particularly easily and cheaply.

Preferably the hygiene mouthpiece element comprises a filter element housing. Preferably the first filter element and the second filter element and/or any further preferably provided filter element are arranged in the filter element housing.

Preferably the filter element housing comprises at least one placement structure, in particular a placement edge and/or placement surface. The placement structure advantageously makes it possible to place the hygiene mouthpiece element on a surface, for example, a table, and prevent the hygiene mouthpiece element from rolling downwards.

The placement structures are preferably arranged circumferentially around the filter element housing so that the filter element housing can be placed on the surface in different orientations.

Preferably the filter housing is configured to be polygonal, in particular triangular, in a cross-sectional view perpendicular to a flow direction through the filter element housing for the provision of a plurality of placement edges and/or placement surfaces. Due to the polygonal configuration, the filter element housing preferably comprises a plurality of housing outer surfaces areas which are configured to be substantially flat or straight, by means of which the hygiene mouthpiece element can be placed in a fixed position on the surface.

Preferably an aroma element is arranged inside the filter element housing. The aroma element is preferably arranged in the filter element housing in such a manner that this comes in contact with air which is flowing past so that the aroma is taken up by the air. Preferably the aroma element is arranged upstream or downstream of the filter elements when viewed in the flow direction of the air. Preferably the aroma element can additionally or alternatively be integrated in one or more of the filter elements.

Further, the object according to the invention is achieved by a handle mouthpiece element comprising a first end section which is configured for connection to a tube of a water pipe and comprising a second end section which is configured as a mouth-side free end and/or which is configured for connection to a hygiene mouthpiece element, in particular to a hygiene mouthpiece element having the previously described features. The handle mouthpiece element comprises a third filter element, wherein the third filter element comprises at least one adsorbing material and/or wherein the handle mouthpiece element comprises a fourth filter element, wherein the fourth filter element comprises metal oxide and/or electrostatically charged fibres.

The handle mouthpiece element preferably is a handle piece component of a water pipe, which in particular during use of the water pipe remains on this and is optionally changed only rarely compared to the hygiene mouthpiece element. In particular, the handle mouthpiece element remains on the water pipe when this is used successively by several persons. In particular, the handle mouthpiece element is not a disposable article.

Preferably, the handle mouthpiece element is configured in such a manner that it can either be used as a mouthpiece alone, i.e., without using a further hygiene mouthpiece, or combined with a hygiene mouthpiece element, in particular a hygiene mouthpiece element having the previously described features. If a hygiene mouthpiece is used, this is preferably placed on the free end of the handle mouthpiece element. If the handle mouthpiece element is used without a hygiene mouthpiece element, the free end of the handle mouthpiece element preferably serves as the mouth-side end of the water pipe.

Preferably the handle mouthpiece element is configured for connection to common water pipes or water pipe tubes. Preferably an adapter can be provided which is arranged between the handle mouthpiece element and the water pipe or the water pipe tube so that the handle mouthpiece element can be advantageously connected to a plurality of water pipes or water pipe tubes.

Preferably the adsorbing material of the third filter element is activated charcoal, in particular activated charcoal fibres. Preferably the activated charcoal is arranged in granular form in the third filter element. Further, it can preferably be provided that the activated charcoal is present in powder form.

However, the adsorbing material of the third filter element can fundamentally comprise any material which is able to adsorb micro-organisms, in particular fungi and bacteria, and viruses. The adsorbing material of the third filter element can, for example, comprise inter alia zeolite or other known adsorbents. In particular, the adsorbing material is capable of adsorbing corona viruses such as SARS-CoV-2.

Preferably the adsorbing material of the third filter element comprises metal oxide. The metal oxide advantageously increases the adsorption efficiency of the third filter element. Preferably the metal oxide is arranged in granular form in the third filter element. Further, preferably an at least partial coating of the activated charcoal with metal oxide can be provided.

In particular preferably, a particularly effective adsorption or filtering of micro-organisms, in particular fungi and bacteria, and viruses in the third filter element can be accomplished by the combination of activated charcoal and metal oxide in the third filter element. Preferably both the activated charcoal and also the metal oxide can be provided in granular form in the third filter element.

Preferably the fourth filter element comprises copper oxide, in particular nanofibres charged with copper oxide. Copper oxide, in particular fibres charged with copper oxide, enables micro-organisms, in particular fungi and bacteria, and viruses, to be killed or deactivated particularly effectively.

Preferably the third filter element and/or the fourth filter element comprise a fibre product, in particular a nanofibre product.

In particular preferably the fourth filter element comprises nanofibres charged with metal oxide, in particular copper oxide. This enables micro-organisms, in particular fungi and bacteria, and viruses to be killed or deactivated particularly effectively.

Fundamentally the third and/or fourth filter element can preferably comprise all materials which can be charged with metal oxide, in particular copper oxide.

Preferably in addition to the third filter element and the fourth filter element, the handle mouthpiece element can comprise further filter elements, wherein the filter elements are preferably arranged one after the other when viewed in the flow direction of the drawn-in smoke. The handle mouthpiece element can preferably comprise three, four or even more filter elements. By providing further filter elements, the filter efficiency can advantageously be increased and arbitrarily adapted. Preferably the further filter elements are configured identically to the third filter element and/or the fourth filter element. Alternatively the further filter elements can be configured differently to the third and/or the fourth filter element.

For example, the handle mouthpiece element can have the third filter element comprising activated charcoal, the fourth filter element comprising electrostatically charged fibres and another filter element comprising metal oxide, in particular copper oxide. Further preferably any other combinations of filter elements and filter technologies are feasible.

Preferably the surface of the handle mouthpiece element, in particular a housing of the handle mouthpiece element, has a coating in some areas or over the entire area. The coating is configured to kill or deactivate micro-organisms, in particular fungi and bacteria and viruses on the surface. Preferably the coating is configured to kill at least 50%, particularly preferably at least 75%, quite particularly preferably at least 99.99%, of the micro-organisms, in particular fungi and bacteria, and viruses located on the surface.

The surface of the handle mouthpiece element preferably comprises all the areas of the handle mouthpiece element facing the surroundings. Particularly preferably the coating is therefore provided on the housing of the handle mouthpiece element in which the third filter element and/or the fourth filter element are arranged. Preferably the surface of the handle mouthpiece element also comprises the surfaces of the interior of the handle mouthpiece element, in particular the interior of the housing of the handle mouthpiece element.

The coating of the surface of the handle mouthpiece element advantageously makes it possible to prevent any transmission of micro-organisms, in particular fungi and bacteria, and viruses, which are present on the hands of a person and which are thus transferred to the surface thereof upon touching the handle mouthpiece element.

The coating can preferably be applied to the surface of the handle mouthpiece element during manufacture of the handle mouthpiece element. Alternatively the coating of the surface of the handle mouthpiece element can preferably also be accomplished subsequently.

Preferably the entire surface of the handle mouthpiece element is provided with a coating. Alternatively only one region of the handle mouthpiece element can have the coating. In particular, it is feasible that, in particular exclusively, a handle area of the handle mouthpiece element can have the coating.

Preferably the coating comprises metal oxide ions. Metal oxide ions are capable of very effectively killing or deactivating micro-organisms, in particular fungi and bacteria, and viruses.

Preferably the third filter element and the fourth filter element are arranged one behind the other in the flow direction of smoke sucked through the water pipe. As a result, preferably a first filtering or adsorption is achieved by means of the third or fourth filter element and subsequently, micro-organisms, in particular fungi and bacteria, and viruses which have not been killed or deactivated by means of the third or fourth filter element, are killed or deactivated by means of the respectively other filter element in a second filtering or adsorption, whereby the efficiency of the filtering in the water pipe is advantageously increased.

Preferably the third filter element is arranged upstream of the fourth filter element when viewed in the flow direction of smoke sucked in by the water pipe. Alternatively the fourth filter element can be arranged upstream of the third filter element when viewed in the flow direction of smoke sucked in by the water pipe.

Preferably the first filter element of the hygiene mouthpiece element and the third filter element of the handle mouthpiece element are configured identically with regard to their filter properties, which means with regard to the arrangement of the adsorbing material and the metal oxide, in particular the form, quantity and arrangement of this inside the respective filter element. Deviations can optionally be given in the overall form of the filter elements so that these can be arranged in the respective components of the water pipe, i.e. the hygiene mouthpiece element or the handle-hygiene mouthpiece element.

The same applies to the configurations of the second and fourth filter element which can also be configured identically in relation to their filter properties.

Preferably the third filter element and/or the second filter element are or is arranged detachably in the handle mouthpiece element. Since in particular the handle mouthpiece element is not designed as a disposable article, it can be advantageous to be able to remove and exchange the third and/or the fourth filter element from the handle mouthpiece element in order to thus prevent the risk of transmission of micro-organisms, in particular fungi and bacteria, and viruses, via the handle mouthpiece element. In particular, if the handle mouthpiece element was used by several persons successively without changing the handle mouthpiece element, it can be appropriate to change the third and/or fourth filter element after a predetermined number of uses.

Preferably for this purpose the housing of the handle mouthpiece element is constructed at least in two parts, particularly preferably in three parts so that the two or three parts of the housing can be detached from one another so that a first receiving region for the third filter element and/or a second receiving region for the fourth filter element are accessible so that the third or fourth filter element can be removed and exchanged from the respective receiving regions.

In the two-part embodiment of the housing the first and the second filter element are preferably arranged together one after the other in a receiving region. Alternatively in the two-part embodiment of the housing the handle mouthpiece element can merely comprise the first or the second filter element so that merely one filter element is then arranged in the receiving region. In the three-part embodiment of the housing, the housing preferably has two receiving regions separated by a housing part so that the first and the second filter element are each arranged in a receiving region.

Preferably the two or three parts of the housing can be detachably connected to one another by means of a screw connection, a locking mechanism, a magnetic connection, a plug-in connection and/or further detachable types of connection.

Alternatively it can preferably be provided that the third and/or further filter element can each be removed through an opening in the housing from the respective receiving region. In this case, the two- or three-part structure of the housing of the handle mouthpiece element is preferably omitted.

Preferably the handle mouthpiece element has a filter element housing. Preferably the third filter element and the fourth filter element and/or each further preferably provided filter element are arranged in the filter element housing.

The filter element housing of the handle mouthpiece element can preferably comprise one or more of the configuration features of the filter element housing of the hygiene mouthpiece element. In particular, the filter element housing of the handle mouthpiece element and the filter element housing of the hygiene mouthpiece element are configured identically. The explanations for the filter element housing of the hygiene mouthpiece element therefore apply similarly at this point.

Furthermore, the object according to the invention is achieved by a mouthpiece device comprising a hygiene mouthpiece element and/or a handle mouthpiece element in each case having the previously described features.

The mouthpiece device can preferably comprise a hygiene mouthpiece element having the previously described features combined with a conventional handle mouthpiece element, that means a handle mouthpiece element without third and/or fourth filter material.

The mouthpiece device can preferably comprise a handle mouthpiece element having the previously described features combined with a conventional hygiene mouthpiece element combined with a conventional hygiene mouthpiece element. The conventional hygiene mouthpiece element preferably comprises a hygiene mouthpiece element without first and/or second filter material.

Preferably further the hygiene mouthpiece element and the handle mouthpiece element can each comprise only one filter element so that, for example, the hygiene mouthpiece element only comprises the first or the second filter element and the handle mouthpiece element only comprises the third or the fourth filter element. As a result, an efficient filter or adsorption by means of two differently acting filter elements is advantageously also achieved without two filter elements needing to be provided in both components. As a result, the manufacture of the components is advantageously easier and cheaper.

In general, preferably all possible combinations of the first, second, third and fourth filter element in the hygiene mouthpiece element and the handle mouthpiece element are possible.

The object according to the invention is further achieved by a tube device for a water pipe, comprising a tube section, wherein the tube section comprises a first tube end section which is configured for connection to a water pipe and wherein the tube section comprises a second tube end section which is configured for connection to a mouthpiece element, wherein the tube device further comprises at least a fifth filter element.

Preferably the fifth filter element comprises at least one adsorbing material.

Preferably the fifth filter element is configured analogously to the first filter element of the hygiene mouthpiece element. In particular, the fifth filter element can comprise some or all the configuration features of the first filter element.

Preferably the tube device comprises a sixth filter element.

Preferably the sixth filter element comprises metal oxide and/or electrostatically charged fibres.

Preferably the sixth filter element is configured analogously to the second filter element of the hygiene mouthpiece element. In particular, the sixth filter element can have some or all the configuration features of the second filter element.

Preferably the tube device comprises a filter element housing. Preferably the fifth filter element and/or the sixth filter element are arranged in the filter element housing.

Preferably the filter element housing is arranged in the first tube end section or in the second tube end section.

Preferably the filter element housing comprises at least one placement structure, in particular a placement edge and/or placement surface. The placement structure advantageously makes it possible to place the hygiene mouthpiece element on a surface, for example, a table and prevent the tube device from rolling downwards.

The placement structures are preferably arranged circumferentially around the filter element housing so that the filter element housing can be placed on the surface in different orientations.

Preferably the filter housing is configured to be polygonal, in particular triangular, in a cross-sectional view perpendicular to a flow direction through the filter element housing for the provision of a plurality of placement edges and/or placement surfaces. Due to the polygonal configuration, the filter element housing preferably comprises a plurality of housing outer surfaces areas which are configured to be substantially flat or straight, by means of which the tube device can be placed in a fixed position on the surface.

Preferably the tube device comprises the mouthpiece element, wherein the tube section, the filter element housing and the mouthpiece element are together formed in one piece. As a result of the one-piece design, the tube device can be exchanged together with the mouthpiece element as a whole and/or designed as a disposable article. As a result, the hygiene can advantageously be improved during use.

Preferably an aroma element is arranged inside the filter element housing. The aroma element is preferably arranged in the filter element housing in such a manner that this comes in contact with air which is flowing past so that the aroma is taken up by the air. Preferably the aroma element is arranged upstream or downstream of the filter elements when viewed in the flow direction of the air. Preferably the aroma element can be additionally or alternatively integrated in one or several of the filter elements.

Preferably the tube device can comprise a tube connection element for connection of the tube device to the water pipe. Preferably the tube connection element can comprise at least a seventh filter element. The seventh filter element can preferably be configured similarly to one of the first to sixth filter elements.

Furthermore the object according to the invention is achieved by a water pipe having a mouthpiece device having the previously described features.

The invention further relates to a water pipe, wherein at least one of the components of the water pipe, in particular a water vessel, a tube, an attachment and/or a mouthpiece device of the water pipe has a coating on its surface in some areas or over the entire area, wherein the coating is configured to kill and/or deactivate micro-organisms, in particular fungi and bacteria, and viruses. Preferably the coating is configured to kill at least 50%, particularly preferably at least 75%, quite particularly preferably at least 99.99%, of the micro-organisms, in particular fungi and bacteria, and viruses located on the surface.

The surface of the at least one component of the water pipe preferably comprises all the areas of the respective component facing the surroundings.

The coating of the surface of the at least one component advantageously makes it possible to prevent any transmission of micro-organisms, in particular fungi and bacteria, and viruses, which are present on the hands of a person and which can thus be transferred when touching the respective surface.

The coating can preferably be applied during manufacture of the respective component. Alternatively the coating of the surface of the respective component can preferably also be accomplished subsequently.

Preferably the entire surface of the respective component is provided with a coating. Alternatively only one region or several regions of the surface of the respective component can have the coating. In particular, it is feasible that, in particular exclusively, handle areas of the respective component have the coating.

Preferably the coating of the respective component comprises metal oxide ions. Metal oxide ions are capable of very effectively killing or deactivating micro-organisms, in particular fungi and bacteria, and viruses.

Preferably the coating of the at least one component is identical to the previously described coating of the hygiene mouthpiece element and/or the handle mouthpiece element.

The coating of the water pipe or components thereof in some areas or over the entire area advantageously makes it possible to severely reduce the risk of transmission of micro-organisms, in particular fungi and bacteria, and viruses through contact of the water pipe by several persons.

In particular, in a preferred embodiment of the water pipe in which this has both a coating of the components and also the hygiene mouthpiece element and handle mouthpiece element according to the invention, as a result of the combination of coating and the provision of preferably at least one or particularly preferably several filter elements, a particularly effective filtering or adsorption or killing/deactivation of micro-organisms, in particular fungi and bacteria, and viruses, is possible.

The invention is explained in detail hereinafter with reference to exemplary embodiments. In the figures in purely schematic view

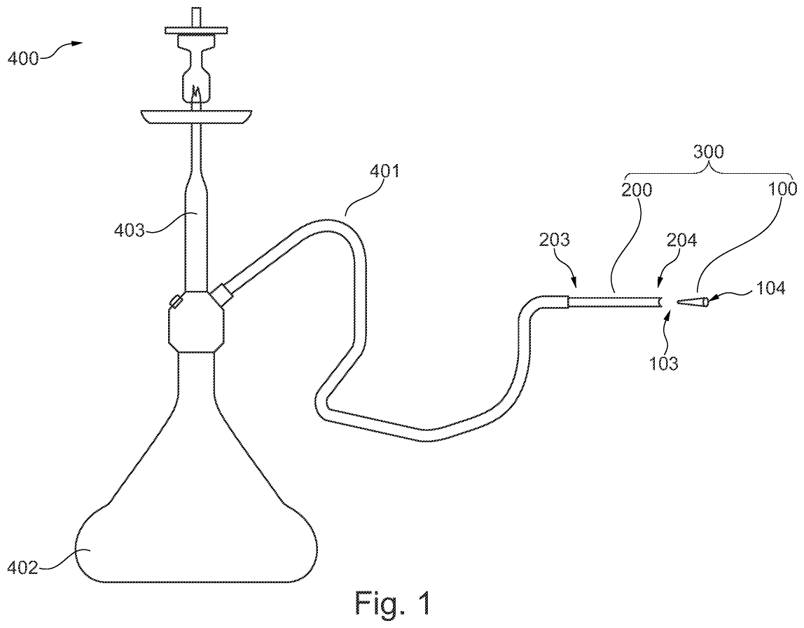

FIG. 1 shows a side view of a water pipe comprising a hygiene mouthpiece element and a handle mouthpiece element,

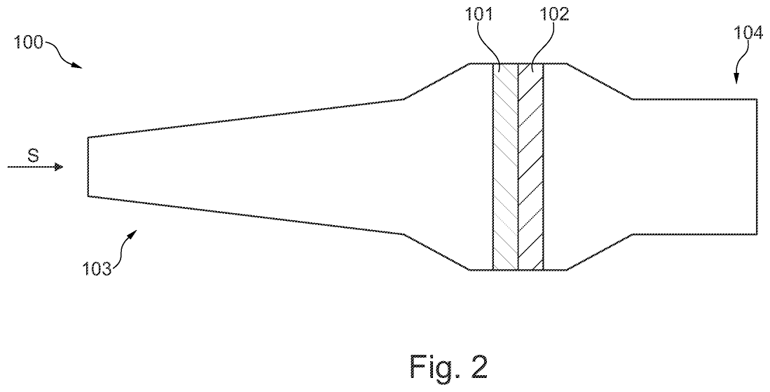

FIG. 2 shows a side view of a handle mouthpiece element,

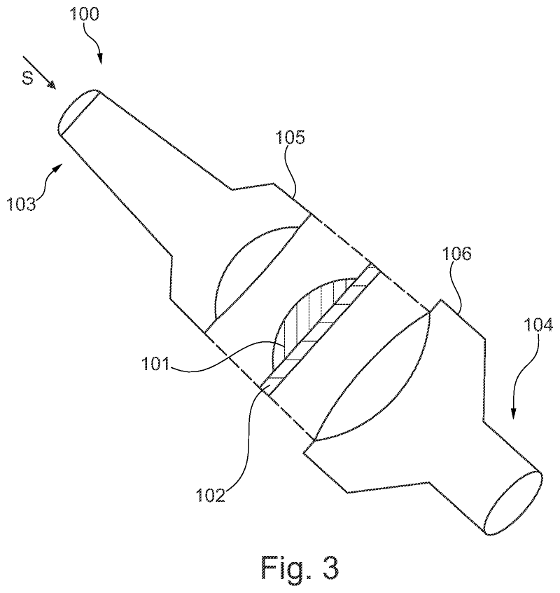

FIG. 3 shows an exploded view of a hygiene mouthpiece element,

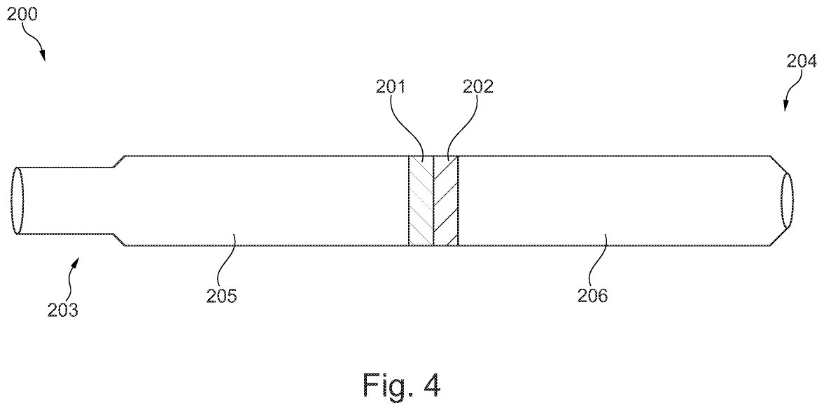

FIG. 4 shows a side view of a handle mouthpiece element according to a first embodiment,

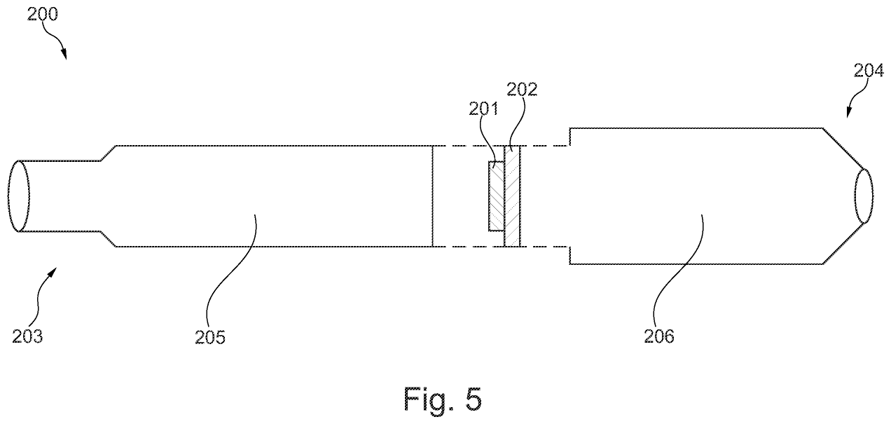

FIG. 5 shows an exploded view of a handle mouthpiece element according to a second embodiment,

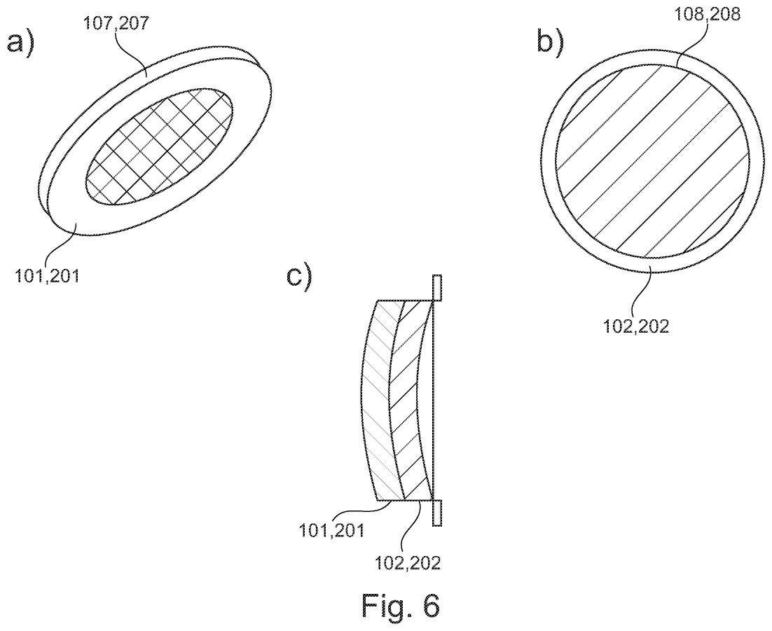

FIG. 6 shows perspective views of two filter elements,

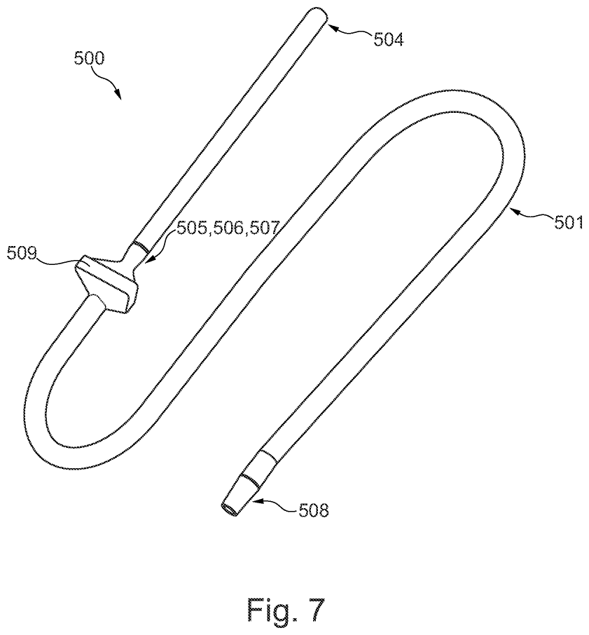

FIG. 7 shows a perspective view of a tube device according to a first embodiment and

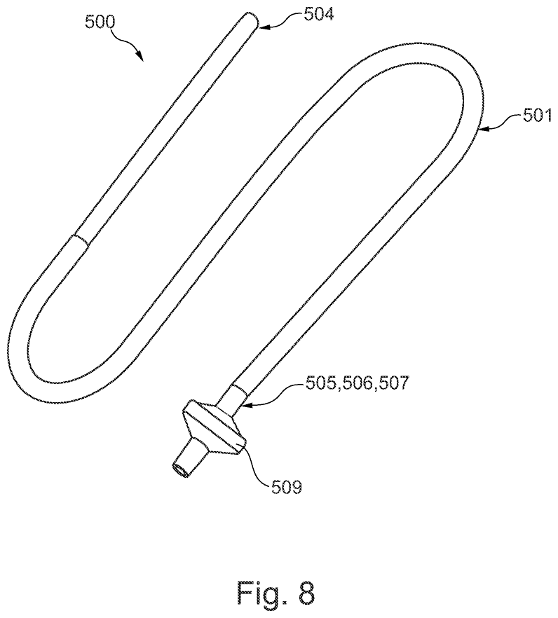

FIG. 8 shows a perspective view of a tube device according to a second embodiment.

FIG. 1 shows a side view of a water pipe 400 comprising a water vessel 402, an attachment 403 arranged thereon and a tube 401. The tube 401 connects a mouthpiece device 300 to the water vessel 402. The mouthpiece device 300 comprises a handle mouthpiece element 200 and a hygiene mouthpiece element 100. In this case, a first end section 203 of the handle mouthpiece element 200 is connected to an end section of the tube 401 and a second end section 204 of the handle mouthpiece element 200 is connected to a first end section 103 of the hygiene mouthpiece element 100.

The water pipe shown in FIG. 1 preferably has a coating on the respective surfaces of the components on all the components, that is on the water pipe 402, the attachment 403, the tube 401 and on the mouthpiece device 300. The coating comprises in particular metal oxide ions.

FIG. 2 shows a side view of the hygiene mouthpiece element 100. The hygiene mouthpiece element 100 has a first end section 104 which can be connected for connection to the second end section 204 of the handle mouthpiece element 200. Further, the hygiene mouthpiece element has a second end section 103 which is configured as a free mouth-side end. In a central region the hygiene mouthpiece element 100 has a first filter element 101 and a second filter element 102.

The first filter element 101 and the second filter element 102 are arranged one after the other when viewed in the flow direction of the smoke S, wherein the second filter element 102 is arranged downstream of the first filter element 101. The first filter element 101 and the second filter element 102 are arranged in the hygiene mouthpiece element 100 in such a manner that they extend over the entire flow cross-section inside the hygiene mouthpiece element 100. The surface of the hygiene mouthpiece element 100 has a coating which comprises metal oxide ions.

FIG. 3 shows the hygiene mouthpiece element 100 in an exploded view. The hygiene mouthpiece element 100 has a two-part housing 105, 106, wherein the first filter element 101 and the second filter element 102 are arranged between the two housing parts 105, 106. Preferably the first filter element 101 and the second filter element 102 are arranged fixedly between the two housing parts 105, 106 so that the latter is configured as a disposable article.

FIG. 4 shows the handle mouthpiece element 200 in a side view. The first end section 203 of the handle mouthpiece element 200 is configured for connection to one end of the tube 401 whilst the second end section 204 is configured for connection to a hygiene mouthpiece element 200. Furthermore, the second end section 204 can also be used as a free mouth-side end of the water pipe 400.

The handle mouthpiece element 200 has a housing comprising two housing parts 205, 206. A third filter element 201 and a fourth filter element 202 are arranged between the two housing parts 205, 206. The surface of the handle mouthpiece element 200 or the surface of the housing parts 205, 206 has or have a coating which comprises metal ions. Preferably the two filter elements 201, 202 in this embodiment are connected firmly to the two housing parts 205, 206 and it is not possible to change the two filter elements. This embodiment is advantageously easy to manufacture and cheap.

FIG. 5 shows a further embodiment of the handle mouthpiece element 200.

The handle mouthpiece element 200 has two housing parts 205, 206 between which a third filter element 201 and a fourth filter element 202 are arranged. The first housing part 205 and the second housing part 206 are connected detachably to one another by means of a plug-in mechanism so that the handle mouthpiece element 200 or the housing thereof can be opened and thus a receiving region for the first filter element 201 and the second filter element 202 is accessible and it is possible to exchange the two filter elements 201, 202.

FIG. 6 shows the first filter element 101, 201 (FIG. 6a)) and the second filter element 102, 202 (FIG. 6b)) individually and in combination (FIG. 6c)). The filter elements 101, 201 are configured as flat fibres or fabric layers which are each held in a frame 107, 207, 108, 208. By means of the frames 107, 207, 108, 208 the two filter elements 101, 102 can be inserted into the hygiene mouthpiece element 100 and/or into the handle mouthpiece element 200 and fastened.

The first filter element 101, 201 comprises activated charcoal and/or metal oxide in its fibre or fabric layer. The metal oxide preferably comprises copper oxide, in particular nanofibres, charged with copper oxide. The second filter element 102, 202 comprises metal oxide and/or electrostatically charged fibres.

FIG. 6c) shows the first filter element 101, 201 combined with the second filter element 102, 202, wherein the two filter elements are arranged one after the other. They can be inserted in any orientation in relation to the flow direction of the sucked-in smoke in the hygiene mouthpiece element 100 and/or in the handle mouthpiece element 200. This means that in relation to the flow direction of the sucked-in smoke, the first filter element 101, 201 can be arranged upstream of the second filter element 201, 202 or conversely.

FIG. 7 shows a tube device 500 according to a first embodiment. The tube device has an elongated tube section 501 on which a mouthpiece element 504 is arranged on the first tube end section thereof and a water pipe not shown here can be arranged on the second tube end section thereof. The connection to the water pipe can be made via a tube connecting element 508.

The tube device 500 has a filter element housing 505 which is arranged on the first tube end section. A first filter element 506 and preferably a second filter element 507 is arranged in the filter element housing 505. The filter element housing (505) is configured to be triangular in a cross-sectional view perpendicular to the flow direction of air sucked in through the tube device 500. Due to the triangular shape, a placement structure 509 with three placement surfaces is formed on an outer side of the filter element housing 505, by means of which the tube device 500 together with the filter element housing 505 can be placed in a fixed position on a surface.

FIG. 8 shows a further embodiment of the tube device 500. The difference from the embodiment shown in FIG. 7 consists in the arrangement of the filter element housing 505 which is arranged on the second tube end section in the embodiment according to FIG. 8.

1-41. (canceled)

42. A hygiene mouthpiece element comprising:
a first end section which is configured for connection to a handle mouthpiece element of a water pipe;
a second end section which is configured as a mouth-side free end, and
wherein the hygiene mouthpiece element includes a first filter element, the first filter element having at least one adsorbing material and/or wherein the hygiene mouthpiece element includes a second filter element, the second filter element having metal oxide and/or electrolytically charged fibres.

43. A hygiene mouthpiece element according to claim 42, wherein the adsorbing material of the first filter element is activated charcoal, in particular activated charcoal fibres and/or wherein the adsorbing material of the first filter element comprises metal oxide.

44. A hygiene mouthpiece element according to claim 42, wherein the second filter element comprises copper oxide.

45. A hygiene mouthpiece element according to claim 42, wherein the first filter element and/or the second filter element comprise a fibre product, in particular a nanofibre product.

46. A hygiene mouthpiece element according to claim 42, wherein the surface of the hygiene mouthpiece element, in particular a housing of the hygiene mouthpiece element, has a coating in some areas or over the entire area, wherein the coating is configured to kill or deactivate micro-organisms, in particular fungi and bacteria, and viruses on the surface, wherein preferably the coating comprises metal oxide ions.

47. A hygiene mouthpiece element according to claim 42 comprising a filter element housing.

48. A hygiene mouthpiece element according to claim 47, wherein the filter element housing comprises at least one placement structure, in particular a placement edge and/or placement surface.

49. A hygiene mouthpiece element according to claim 48, wherein the filter housing is configured to be polygonal, in particular triangular, in a cross-sectional view perpendicular to a flow direction through the filter element housing for the provision of a plurality of placement edges and/or placement surfaces.

50. A hygiene mouthpiece element according to claim 47, wherein an aroma element is arranged inside the filter element housing.

51. A handle mouthpiece element comprising:
a first end section which is configured for connection to a tube of a water pipe;
a second end section which is configured as a mouth-side free end and/or which is configured for connection to a hygiene mouthpiece element wherein the handle mouthpiece element includes a third filter element, the third filter element having at least one adsorbing material and/or wherein the handle mouthpiece element includes a fourth filter element, the fourth filter element having metal oxide and/or electrostatically charged fibres.

52. A handle mouthpiece element according to claim 51, wherein the adsorbing material of the third filter element is activated charcoal, in particular activated charcoal fibres and/or wherein the adsorbing material of the third filter element comprises metal oxide.

53. A handle mouthpiece element according to claim 51, wherein the fourth filter element comprises copper oxide.

54. A handle mouthpiece element according to claim 51, wherein the third filter element and/or the fourth filter element comprise a fibre product, in particular a nanofibre product.

55. A handle mouthpiece element according to 51, wherein the surface of the handle mouthpiece element, in particular a housing of the handle mouthpiece element, has a coating in some areas or over the entire area, wherein the coating is configured to kill or deactivate micro-organisms, in particular fungi and bacteria, and viruses, wherein preferably the coating comprises metal oxide, in particular copper oxide and/or metal oxide ions.

56. A handle mouthpiece element according to claim 55, wherein the handle mouthpiece element has a handle region, in particular on the housing of the handle mouthpiece element and wherein the handle region has the coating.

57. A handle mouthpiece element according to claim 51, comprising a filter element housing.

58. A handle mouthpiece element according to claim 57, wherein the filter element housing comprises at least one placement structure, in particular a placement edge and/or placement surface.

59. A handle mouthpiece element according to claim 58, wherein the filter housing is configured to be polygonal, in particular triangular, in a cross-sectional view perpendicular to a flow direction through the filter element housing for the provision of a plurality of placement edges and/or placement surfaces.

60. A handle mouthpiece element according to claim 57, wherein an aroma element is arranged inside the filter element housing.

61. A water pipe comprising:
a hygiene mouthpiece element having a first end section which is configured for connection to a handle mouthpiece element of a water pipe;
a second end section which is configured as a mouth-side free end, wherein the hygiene mouthpiece element includes first filter element, the first filter element having at least one adsorbing material and/or wherein the hygiene mouthpiece element includes a second filter element, the second filter element having metal oxide and/or electrolytically charged fibres and/or handle mouthpiece element comprising a first end section which is configured for connection to a tube of a water pipe and comprising a second end section which is configured as a mouth-side free end and/or which is configured for connection to a hygiene mouthpiece element wherein the handle mouthpiece element includes a third filter element, the third filter element having at least one adsorbing material and/or wherein the handle mouthpiece element includes a fourth filter element, the fourth filter element having metal oxide and/or electrostatically charged fibres.

62. A water pipe according to claim 61, comprising a tube device for a water pipe, comprising a tube section, wherein the tube section comprises a first tube end section which is configured for connection to a water pipe and wherein the tube section comprises a second tube end section which is configured for connection to a mouthpiece element, wherein the tube device further comprises at least a fifth filter element.
